(19) United States
(12) Patent Application Publication
(10) Pub. No.: US 2020/0324076 A1
(43) Pub. Date: Oct. 15, 2020

(54) RESTORATIVE MUSICAL METHOD AND SYSTEM

(71) Applicant: Pineal Labs LLC, New York, NY (US)

(72) Inventors: Neal Sarin, New York, NY (US); Warren Riker, Rotunda West, FL (US)

(21) Appl. No.: 16/914,159

(22) Filed: Jun. 26, 2020

Related U.S. Application Data
(63) Continuation-in-part of application No. 16/879,580, filed on May 20, 2020.

(51) Int. Cl. A61M 21/02; G06F 3/16; H04R 5/033; H04R 5/04; H04S 7/00

(52) U.S. Cl. CPC A61M 21/02; G06F 3/165; H04R 5/033; A61M 2021/0027; H04S 7/304; H04S 2400/11; H04S 2400/13; H04R 5/04

(57) ABSTRACT

A restorative musical system having a musical composition, which has a plurality of audio elements, a start and an end, and a slow tempo. The plurality of audio elements having a repeating phrase, an instrumentation of soft tones and a pulsating sound, a plurality of arrangements, and a field recording element. The plurality of arrangements and the instrumentation are adapted to be introduced in succession, while the phrase is adapted to repeat within the musical composition. Additionally, the plurality of audio elements is audio mixed to emphasize solfeggio frequencies and are adapted to cause moving stereo imaging.

### CROSS-REFERENCE TO RELATED APPLICATIONS

This application is a continuation-in-part and claims the benefit of U.S. Non-Provisional application Ser. No. 16/879,580, filed May 20, 2020, which is hereby incorporated by reference, to the extent that it is not conflicting with the present application.

### BACKGROUND OF INVENTION

### 1. Field of the Invention

The invention relates generally to the composition of music and more specifically to a stress relieving musical composition.

### 2. Description of the Related Art

A majority of US workers suffer from work-related stress among other stressors throughout their daily lives. It is known that stress contributes to major health problems including cardiovascular and psychiatric disease. The World Health Organization predicts that stress and anxiety will be the leading cause of disease in young people in 2020. Some common treatments for stress and anxiety are meditation, medication, a variety of lifestyle choices such as eating healthy and exercising. However, these solutions take an incredible amount of time, investment and discipline. Also, these methods of stress reduction do not provide instant relief.

Additionally, solutions such as medication may have unwelcome side effects causing more issues for the person. For people who have not mastered meditation, a quiet environment is necessary to receive the stress relieving benefits. Meditation is also something that typically requires training and is not a natural skill that most people acquire.

Therefore, there is a need to solve the problems described above by proving a more intuitive, universal and cost-effective system and method for reducing stress.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an aspect, a restorative musical system having a musical composition with a plurality of audio elements, a start and an end, and a slow tempo. The audio elements are a musical phrase adapted to repeat within the musical composition, an instrumentation including soft tones and pulsating sounds, a plurality of arrangements, and a field recording element. The audio elements are organized to form a melody. Furthermore, audio mixing the audio elements allows the musical composition to have moving stereo imaging. The combination of the audio elements of the musical composition engages the listener's attention, resulting in a calm and relaxed mental state. Thus, an advantage is stress relief for the listener. Another advantage is more time-efficient stress relief, since listening to the musical composition and its stress relieving effects are simultaneous.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 illustrates the musical composition, according to an aspect.

FIG. 2 illustrates a graphic of the participant's responses to the research study's questions.

FIG. 3 illustrates a table of the research study's data.

### DETAILED DESCRIPTION

What follows is a description of various aspects, embodiments and/or examples in which the invention may be practiced. The aspects, embodiments and/or examples described herein are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

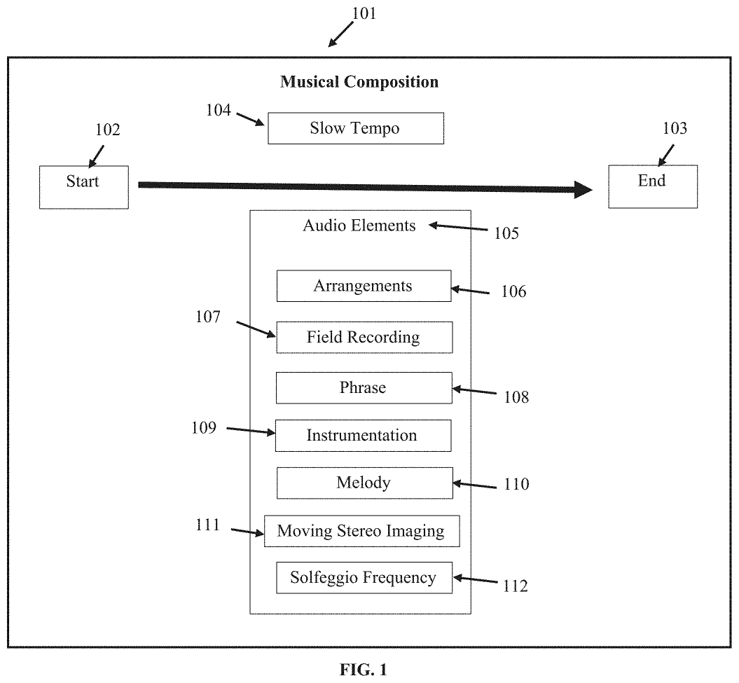

A restorative musical system containing a combination of musical elements (“audio elements”) such as repetition, melody, slow tempo, a gradual build in arrangements, solfeggio frequencies, field recordings, and instrumentation, played through a music player, to create a musical composition that allows a listener to relax, thus reducing the user's stress levels is provided. As shown in FIG. 1 and as it will be described in more detail hereinafter, the combination of these audio elements 105 may work together to increase relaxation in the human body and decrease side effects of high stress levels. For example, the restorative musical composition may further allow the listener to have better focus once in their relaxed state. The restorative musical composition may be played audibly through a sound source, such as a speaker. For example, the restorative musical composition may be played on a speaker or surround sound system. In another example, the restorative musical composition may be played through headphones for convenient listening at any time.

Furthermore, the restorative musical system being a professional sound recording and music composition that incorporates a variety of compositional and mixing techniques, which yields meditative benefits for the listener. The compositional techniques applied being: using the elements of repetition, instrumentation, a slow tempo, arrangement progression, use of melody, and field recordings. The mixing techniques applied being 3D imaging and the equalization (EQ) of solfeggio frequencies.

Typically, stress is managed by meditation, medication, a variety of lifestyle choices such as eating healthy and exercising. However, as all of these may help reduce stress and anxiety in the average person these solutions take an incredible amount of time, investment and discipline.

The restorative musical system having multiple musical elements, the elements being repetition, successional arrangements, melody, instrumentation, field recordings, tempo, moving stereo imaging, and solfeggio frequencies. The restorative musical system, when listened to by a person, reduces stress in said person because of the combination of musical elements creating the restorative musical system. The restorative musical system is used as a method of treatment for stress as will be described herein.

The restorative musical composition having a phrase (“musical phrase”) repeated within the musical composition, for example, such as a chord progression. The phrase may also be a melody. Typically, the use of repetition results in brainwave entrainment, also referred to as neural entrainment. This denotes the way in which the aggregate frequency of oscillations produced by the synchronous electrical activity in ensembles of cortical neurons can adjust to synchronize with the periodic vibration of an external stimuli, such as a sustained acoustic frequency (perceived as pitch), or a regularly repeating pattern of intermittent sounds (perceived as rhythm). It is believed that patterns of neural firing, measured in Hertz (Hz), correspond with states of alertness such as focused attention and deep sleep. The repetition of the phrase may allow the listener to be more engaged with the musical composition and serve as an anchor to hold the listener's attention. For example, the repetition of the musical phrase may act as a mantra phrase (“mantra”) in traditional meditation methods. Typically, a mantra is repeated throughout a meditation to keep the person anchored and focused on the meditation instead of letting their mind wander. The repetition of the phrase may also act as an anchor for the listener's focus, which continuously brings the listener's focus back to the restorative musical composition. In an example, the repetition of the phrase may slow or increase in pace to bring the listener's focus in and out, which may allow for a longer meditative state and may give-the listener a more fluid listening experience.

The restorative musical composition also having a slow tempo. The tempo of a song refers to the speed at which a piece of music is or should be played. Additionally, studies have shown that a song's tempo can influence the listener's heart rate. The restorative musical composition plays at a slower pace to reflect a resting heart rate. For example, the restorative music composition may have a tempo range of between 40 and 65 beats per minute to reflect the slower heart rate pace. A higher heart rate typically indicates a stressed or active state; thus, the slow tempo encourages a slowing heart rate and reduces stress. The slow tempo in combination with the other elements of the musical composition further facilitates relaxation for the listener.

The restorative musical composition having arrangements added in progression to draw the listener into the composition. The addition of each arrangement in a progression draws the listener in by introducing individual audio elements in a succession. This means not all of the audio elements of the musical composition begin at the same time, such as the introduction of the repeating phrase then the addition of a field recording sound. There may be a gradual buildup of the audio elements, which create a more relaxing listening environment. In another example, audio elements may be introduced simultaneously in a way that does not disturb or distract the listener, such as each audio element being introduced at a different volume level, with the gradual succession of balancing the volume of each audio element in different ways. The plurality of arrangements introduced in succession engage the listener through a gradual buildup and release of sound. For example, the arrangement may begin with a repeating phrase upon which the musical composition can be built.

Introducing each arrangement in succession also does not interfere with the listener's focus on the anchor but creates a more enjoyable listening environment. Beginning the composition with the phrase arrangement allows the listener to be drawn in slowly thus keeping the listener engaged with the musical composition. Having the listener engaged while listening to the entire composition further allows the listener to focus on relaxing throughout the entire musical composition.

The restorative musical composition also having an instrumentation of soft tones. A soft tone refers to sound sources which tend to have longer attack and/or release times and quieter transients. This helps soften the envelope's characteristics, resulting in a more pleasant and less abrasive subjective tone. The restorative musical composition may also have the instrumentation of pulsating sounds. For example, the instrumentation may utilize synths, percussion, guitar, and other musical elements including synthetic and natural instruments and sounds. The pulsating sound is defined by a soft tone being turned up and down turned in volume, like a pulse. The pulsating sound is subliminally or evidently used in the musical composition to achieve stress-reducing benefits.

The restorative musical composition also having a melody. The melody allows the musical composition to be distinguishable compared to other pieces of music by making it more memorable to the listener. Melodies are pleasing sequences of musical notes, which typically allow a listener to enjoy the composition. The melody is used in the musical composition to engage the listener in a pleasing and novel activity rather than only providing a form of stress relieving treatment. The melody may also increase commercial appeal because it is musically satisfying. For example, without a distinct melody, the listener may not find the musical composition to be as memorable. The melody may ensure the musical composition is pleasant and unique sounding to the listener with all the necessary stress relieving elements.

The restorative musical composition may also utilize moving stereo imaging. Stereo imaging is the perceived spatial location of the sound source, both laterally and in depth, with each sound produced to play from a specific location. While moving stereo imaging allows the audio elements to have movement and fluidly flow from different sound sources. Moving stereo imaging allows each individual audio element, sound, or instrument during the musical composition to create a multidimensional (3D) experience for the listener, in which various sonic elements move in a way that stimulates different hemispheres of the brain. For example, the moving stereo imaging may produce a Doppler effect, which is frequency and volume changes as a sound source travels across the stereo field. The moving stereo imaging may also have custom phase oscillated movements, and multi binaural stereo and comb filtering to create the depths and perceptions of each audio element. The moving stereo imaging further allows the audio elements to sound as if each audio element is moving through the stereo field.

In addition, the moving stereo imaging allows the listener to have a dynamic sound experience, which may help stimulate and focus the listener's brain while engaging with the musical composition. The moving stereo imaging may allow the sounds of the musical composition to slowly move from one sound source to another, such as from one speaker to another speaker. In another example, the musical composition may play from one ear to the other while the listener was using headphones.

The restorative musical composition having audio elements with emphasized solfeggio frequencies. Solfeggio frequencies are specific tones of sound that help and promote various aspects of body and mind health. These solfeggio frequencies are known in the art and are fundamental sounds used in both Western Christianity and Eastern Indian religions. The audio elements of the musical composition are audio mixed to amplify the resonance of a solfeggio frequency, thus emphasizing the solfeggio frequency. For example, the audio elements of the musical composition may be audio mixed to play at a 500 Hz to 600 Hz range, which is a common solfeggio frequency range. In another example, the solfeggio frequencies may be boosted using equalization (EQ) on the various audio elements of the musical composition. Equalization is adjusting the frequency of audio elements in a composition by enhancing the frequency in a certain place on the stereo field.

Another audio element of the musical composition is the use of field recordings. The field recording element may soften the musical composition, so it is not too synthetic sounding to the listener. Field recordings are audio recordings produced outside a recording studio and apply to recordings of both natural and human made sounds. Field recordings may be used to add an organic quality to the composition to balance predominantly synthetic-based musical composition. Some examples of field recording elements include sounds of the ocean, birds, a running stream or river, rustling leaves, gentle breathing. The addition of the field recording element may further make the music composition more enjoyable for listeners. The field recordings also provide a natural element to further help the listener relax. Nature sounds are known to help reduce stress in its listener by relaxing their mind.

The restorative musical system having a music player, a speaker, and a musical composition, as shown in FIG. 1. The musical composition 101, as described herein is a plurality of audio elements 105 with a start 102 and an end 103, and a slow tempo 104. The plurality of audio elements 105 are elements such as a phrase 108, a plurality of arrangements 106, instrumentation 109, a field recording element 107, and a melody 110. The instrumentation 109 having soft tones and may also have a pulsating sound. The phrase is adapted to repeat in the musical composition, while each of the plurality of arrangements of the musical composition are adapted to be introduced in succession. The instrumentation 109 may also be adapted to be introduced in succession. In an example, the instrumentation 109 may be adapted to repeat from the start to the end of the musical composition with few changes in performance. The plurality of audio elements is disposed to form a melody. The plurality of audio elements is adapted to be audio mixed to emphasize a solfeggio frequency 112. The plurality of audio elements is also adapted to cause moving stereo imaging 111. Furthermore, the musical composition is adapted to be played by the music player and be audible through the speaker. The combination of each audio element in the restorative music composition allows the listener to experience the benefits of stress relief. The elements work cohesively to create a listening environment that promotes relaxation. Additionally, it is known that once someone is more relaxed, they can focus better.

Furthermore, the restorative music system as described herein is a method of treatment for stress. As mentioned, stress is a growing problem for personal health. The restorative music system may be listened to by a user to help reduce their stress levels and further improve their everyday health and wellbeing since stress can cause a multitude of other health problems. Furthermore, this method of treatment may be used at any time, such as while working or engaging in sporting activities.

A research study was conducted to evaluate the benefits of the method and system described herein. The study was conducted at the Nielsen Consumer Neuroscience San Francisco location and consisted of sixty-four participants (50% male and 50% female) between the ages of 21 and 34. The experiment group of 32 participants listened to ten minutes of the restorative musical composition, while an equivalent control group of 32 participants went through the same protocol, except that they listened to ten minutes of music that was not designed to reduce stress.

The results of the study concluded the restorative music system led to a 23 percent reduction in reported stress levels. The results of the study also concluded that compared to the control group, the restorative musical composition also succeeded in having a ‘restorative effect’ on the participants' brain state. During the ten minutes of the restorative musical composition, the experimental group participants showed a 13 percent increase in EEG markers of memory activation, specifically increases in theta and gamma band brain oscillations. This data was gathered to see if the music also stimulates the memory cortex of the brain. Memory activation can be defined as the formation of connections with new and past experiences in the amygdala. Additionally, as the experimental group participants listened to the restorative musical composition, they exhibited greater attentional focus as indexed by a decrease in alpha band EEG activity. After the experimental group participants stopped listening to the restorative musical composition, the experimental group participants showed an overall decrease in attention processing, measured by increases in alpha activity. The results of the study showed an overall decrease in attention processing which yielded a calming effect on the listeners.

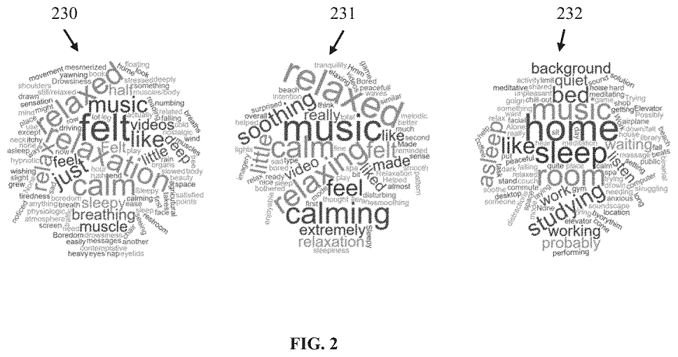

The participants were asked to respond to the following open-ended questions after listening to the musical composition: what physiological sensations did you experience while listening, what's your initial reaction after listening to the music, and in what environment do you see yourself listening to this music. As shown in FIG. 2, the physiological sensations 230 participants responded with were relaxed, relaxation, and calm, while their initial reaction 231 was similar with words such as relaxed, calming, and relaxing. The environments 232 the participants saw themselves listening to the musical composition most were at home, in bed, and while studying. Attentional processes have been extensively linked to meditation and stress relief in previous research studies. Moreover, lower levels of attention, as indexed by higher alpha band EEG activity, are known to reflect states of calm and relaxation. The current results suggest that the restorative music system is both memorable and has a meditative effect on the listener, with more focused attention during the experience followed by greater relaxation.

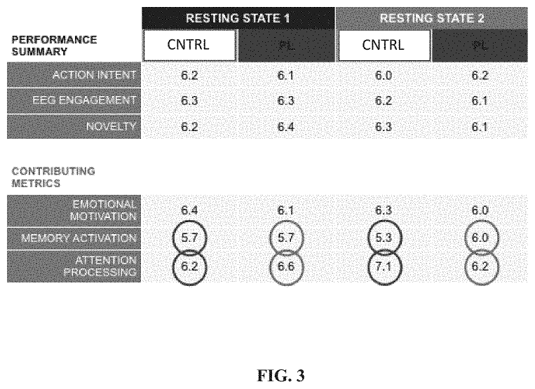

FIG. 3 illustrates a table of the research study's data, which reflects the averaged EEG data collected before and after the 10-minute listening sessions. The experimental group is labeled “PL” and the control group is labeled “CNTRL.” The findings of the research study in FIG. 3 are reported on a 10-point scale based on the EEG results. The “resting state 1,” as shown, is the participants score prior to listening to their assigned music for 10 minutes, while “resting state 2” is the participants score after listening. As shown in FIG. 3, while in the resting state 2 the musical composition leads to lower attention and higher memory, implying a relaxing and memorable experience. As shown, the PL columns from resting state 1 compared to resting state 2 had an increase of memory activation of 0.3 and a decrease of attention processing of 0.4, while the CNTRL had a decrease of memory activation of 0.4 and an increase of attention processing of 0.9 after listening to the musical composition.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims.

If present, use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the aspects, embodiments or examples shown should be considered as exemplars, rather than limitations on the apparatus or procedures disclosed or claimed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

If means-plus-function limitations are recited in the claims, the means are not intended to be limited to the means disclosed in this application for performing the recited function, but are intended to cover in scope any equivalent means, known now or later developed, for performing the recited function.

Claim limitations should be construed as means-plus-function limitations only if the claim recites the term “means” in association with a recited function.

If any presented, the claims directed to a method and/or process should not be limited to the performance of their steps in the order written, and one skilled in the art can readily appreciate that the sequences may be varied and still remain within the spirit and scope of the present invention.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples. Hence, the scope of the invention is defined by the accompanying claims and their equivalents. Further, each and every claim is incorporated as further disclosure into the specification.

What is claimed is:

1. A restorative musical system comprising:
a music player;
a speaker; and
a musical composition, having:
a plurality of audio elements, a start and an end, and a slow tempo;
the plurality of audio elements being:
a phrase adapted to repeat within the musical composition;
a plurality of arrangements, wherein each of the plurality of arrangements is adapted to be introduced in succession;
an instrumentation of soft tones adapted to be introduced in succession;
a field recording element;
wherein the plurality of audio elements is disposed to form a melody;
wherein the plurality of audio elements is adapted to be audio mixed to emphasize solfeggio frequencies, and wherein the plurality of audio elements is adapted to cause moving stereo imaging; and
wherein the musical composition is adapted to be played by the music player and be audible through the speaker.

2. The restorative musical system of claim 1, wherein the field recording element is an ocean sound.

3. The restorative musical system of claim 1, wherein the phrase is a chord.

4. The restorative musical system of claim 1, wherein the solfeggio frequencies are between 500 Hz and 600 Hz.

5. The restorative musical system of claim 1, wherein the slow tempo is between 40 and 65 beats per minute.

6. The restorative musical system of claim 1, wherein each of the plurality of arrangements are a different instrument.

7. The restorative musical system of claim 1, wherein the phrase is adapted to repeat from the start to the end of the musical composition.

8. The restorative musical system of claim 1, wherein the instrumentation has a pulsating sound.

9. A restorative musical system comprising:
a musical composition, having:
a plurality of audio elements, a start and an end, and a slow tempo;
the plurality of audio elements being:
a phrase adapted to repeat within the musical composition;
a plurality of arrangements, wherein each of the plurality of arrangements is adapted to be introduced in succession;
an instrumentation of pulsating sound adapted to be introduced in succession;
a field recording element;
wherein the plurality of audio elements is adapted to be audio mixed to emphasize solfeggio frequencies, and wherein the plurality of audio elements is adapted to cause moving stereo imaging.

10. The restorative musical system of claim 9, wherein the plurality of audio elements organized to form a melody.

11. The restorative musical system of claim 9, wherein the phrase is a melody.

12. The restorative musical system of claim 9, wherein the field recording element is a rustling leaves sound.

13. The restorative musical system of claim 9, wherein the instrumentation are soft tones.

14. The restorative musical system of claim 9, wherein the slow tempo is between 40 and 65 beats per minute.

15. The restorative musical system of claim 9, wherein the pulsating sound is adapted to repeat from the start to the end of the musical composition.

16. A method of treatment of stress comprising the steps of:
receiving a restorative musical system, having a musical composition, the musical composition having:
a plurality of audio elements, a start and an end, and a slow tempo;
the plurality of audio elements being:
a phrase adapted to repeat within the musical composition;
a plurality of arrangements, wherein each of the plurality of arrangements is adapted to be introduced in succession;
an instrumentation of soft tones adapted to be introduced in succession;
wherein the plurality of audio elements is disposed to form a melody;
wherein the plurality of audio elements is adapted to cause moving stereo imaging;
initiating the restorative musical system to play audibly; and
listening to the restorative musical system to reduce stress level.

17. The method of claim 16, wherein the user is listening to the restorative musical system through headphones.
